(19) United States
(12) Patent Application Publication
(10) Pub. No.: US 2020/0268483 A1
(43) Pub. Date: Aug. 27, 2020

(54) MICRO APPLICATOR FOR USE WITH DENTAL ADHESIVES

(71) Applicant: Kulzer GmbH, Hanau (DE)

(72) Inventors: Mike Kondziela, Weilburg (DE); Andrea Kemeny, Usingen (DE)

(73) Assignee: Kulzer GmbH, Hanau (DE)

(21) Appl. No.: 16/753,695

(22) PCT Filed: Oct. 1, 2018
(86) PCT No.: PCT/EP2018/076603

§ 371 Date: Apr. 3, 2020

(30) Foreign Application Priority Data
Oct. 4, 2017 (DE) 10 2017 122 990.6

(51) Int. Cl. A61C 5/64; A61C 3/00; A61C 5/66; A61K 6/61

(52) U.S. Cl. CPC A61C 5/64; A61K 6/61; A61C 5/66; A61C 3/005

(57) ABSTRACT

The invention relates to a dental micro-applicator (1) for applying a liquid or paste in a cavity of a natural tooth, having a hollow sleeve-like application head (2) with a distal (2.2) and a proximal region (2.1) and with an application region (5.1) at the front face of the proximal region (2.1) of the application head (2), a punching spike (4), the tip of which is oriented toward distal, being arranged in the hollow sleeve-like application head (2) in the proximal region (2.1), and an opening (7) being formed at the front face of the proximal region (2.1) for passage of a liquid or paste to be applied, a micro-container (3) being inserted, in particular slid in or turned in, from distal into the hollow sleeve-like application head (2), the outer shape of which being formed in fit to the inner shape of the application head (2), and the micro-container (3) having an internal space (10) to receive the liquid or paste to be applied. Moreover, the micro-container is closed at its distal side (9) and at its proximal front face (6).

The invention relates to a dental micro-applicator for applying a liquid or paste in a cavity of a natural tooth, having a hollow sleeve-like application head with a distal and a proximal region and with an application region at the front face at the proximal region of the application head, wherein a punching spike, the tip of which is oriented toward distal, is arranged in the hollow sleeve-like application head in the proximal region, and an opening is formed at the front face at the proximal region for passage of a liquid or paste to be applied, wherein a micro-container is inserted, in particular slid in or turned in, from distal into the hollow sleeve-like application head, the outer shape of which is formed in fit to the inner shape of the application head, and wherein the micro-container, in particular the tubular micro-container, has an internal space to receive the liquid or paste to be applied. Moreover, the micro-container is closed at its distal side and at its proximal front face.

In order to improve adhesion of dental materials to teeth, a wide variety of bonding and primer combinations are known. Younger generation bonding agents comprise single-component self-etching adhesives. A large easily wettable bonding surface must be provided to ensure good adhesion to the tooth. The adhesive itself must be highly fluid, have good wetting properties and cure very quickly to minimize the possibility of contact with moisture, resulting in reduced adhesion.

Thus, self-etching adhesives are known, combining the steps of acid etching and subsequent application of the adhesive in one step. The acidic adhesive system dissolves the smear layer and exposes the underlying dentin or simply dissolves the smear layer in part to make it permeable to the adhesive ingredients. Simultaneously, the monomers infiltrates into the tooth structure. In the case of enamel, the acidic adhesive systems create an etching pattern similar to that of phosphoric acid etching. The solvent required for infiltration is removed by an air blowing device and the adhesive is cured, e.g. radiation cured. Similarly, desensitizing agents are applied on the tooth or in tooth cavities.

Furthermore, single-component applicators are known, having a reservoir in the body of the applicator that extends continuously until the application medium at the proximal end of the body, as in WO02/30314A1. EP1262151A1 discloses alternative applicators, additionally comprising a separate application element.

There is a need for micro-applicators which can be activated with one hand and are immediately ready for use. In addition, it is the object to provide a micro-applicator directly comprising an application element and being dimensioned in such a way that the applicator or at least the application element can be inserted into the cavity to be treated of a patient's natural tooth. Therefore, the object of the present invention was to develop a micro-applicator which can be used one-handed directly in the cavity of a natural tooth. A particular problem in the development of such a micro-applicator, which is to be able to be inserted into the cavity of a tooth, lies in the requirements for the tightness of all components despite the necessary low material thicknesses as well as the small dimensions of the individual components. In this respect, the exact dimensioning and especially the choice of material is of great importance.

The object is solved by a dental micro-applicator according to claim 1 as well as a kit according to claim 12.

A subject matter of the invention is a dental micro-applicator having a hollow sleeve-like application head which has a distal and a proximal region as well as an application region at the front face of the proximal region, wherein at least one punching spike, the tip of which is oriented toward distal, is arranged, in particular fixed, in the hollow sleeve-like application head. Moreover, an opening, in particular having a cross section (d₁), is formed at the front face of the proximal region for passage of a liquid or paste to be applied, wherein a micro-container, the outer shape of which is formed, in particular in fit, to the inner shape of the application head, in particular in transition fit or press fit, is inserted, in particular a tubular micro container, is slid in or turned in, from distal into the hollow sleeve-like application head, and wherein the micro container, in particular the tubular micro-container, has an internal space, in particular having a volume of less than 1 ml, to receive the liquid or paste to be applied, and wherein the micro-container is closed at its distal side and at its proximal front face. According to a preferred alternative, the outer shape of the tubular micro container is formed in fit to the inner shape of the application head. Cross section (d₁) of the opening of the application head is less than, equal to or greater than cross section (d₄) in the micro-container, in particular the cross sections are approximately equal. Preferably, the micro-container comprises a punching spike, in particular with a conduit. The at least one punching spike may have several conduits, wherein the cross section or the total cross section of the conduits preferably is less than, equal to or greater than the cross section of the opening of the application head. The micro-container may also have several punching spikes in the proximal region in the application head, which are arranged around the opening in the proximal wall of the application head and the tips of which are oriented toward distal. The micro-applicator may comprise in alternatives 2 to 20 punching spikes with or without conduit.

In particular, the outer shape of the micro-container corresponds to the positive of the inner shape of the application head being formed as the negative.

In this context, two alternative embodiments are preferred, according to which a) the micro-container being inserted, in particular slid in or turned in, from distal into the hollow sleeve-like application head, has an outer shape being in fit to the inner shape of the application head, or b) the outer shape of the micro-container is spaced by a seal to the inner shape of the application head. The seal may be formed inside the application head in the distal region as well as also until the proximal region. Alternatively, the seal may be attached to the outside of the micro-container. The seal may be made of a flexible or elastic material, such as, by way of example, polyurethane, silicone or a mixture of elastic plastics.

In this context, the micro-container of the applicator for applying the liquid or paste, in which the inner shape of the hollow sleeve-like application head is formed in fit to the outer shape of the micro-container, in particular the tubular micro-container, may be slid in or turned in, preferably slid in, into the application head until the proximal front face, in particular as front face of the side walls of the micro-container or the film or the fragile layer of the micro-container, in particular of the tubular micro-container, abuts in the inside against the base of the punching spike or against the proximal inside wall of the application head. A preferred embodiment of turning in is screwing in. The film or fragile layer at the proximal side of the micro-container, in particular of the tubular micro container, is destroyed, in particular pierced or cut off, by the punching spike when sliding in or turning in the micro-container into the application head. In this context, the term destroying comprises both cutting off the film or the layer and piercing. The micro-container and the internal cross section of the side wall of the micro-container as well as the external cross section of the punching spike are designed in such a way that the micro-container is slidable at the outside over the punching spike; in particular the punching spike has a conduit, preferably running through a base. The afore-mentioned fit ensures that the transition between the micro-container and the application head is sealed and no liquid can escape at this place. The liquid or paste to be applied escapes from the internal space through the opening in the proximal region of the application head. The micro-container may also have a multitude of punching spikes so that all remarks also apply to these punching spikes. In addition, the respective punching spike may have one or more conduits so that the remarks for one channel also apply to these channels.

The micro-container preferably is a hollow section closed at the distal front face and at the proximal front face. Preferred is a) a tubular micro-container, or b) the micro-container has a hollow section not having a circular cross section, or c) the hollow section has an elliptic, oval, triangular or rectangular cross section as well as appropriate cross sections lying in-between. The hollow sleeve-like application head is preferably formed in fit to the outer shape of the micro-container, in particular of the tubular micro-container or of the micro-container with the afore-mentioned hollow sections. The micro-container may have an external thread or an internal thread and the application head may have the appropriate opposite thread selected from internal and external thread. Alternatively, the micro-container and/or the application head may have at least one guiding groove, in particular an I- or L-shaped guiding groove as well as a lug engaging therein.

According to the invention, the dimensioning of the dental micro-applicator in the application region of the application head in the proximal region is adjusted such that the application region and preferably the application head may be inserted into a cavity of a natural tooth. A cavity is understood to mean a drilled cavity in the natural tooth. According to the invention, the application region and preferably the application head of the micro-applicator should be insertable into the cavity past the cavity edge. Furthermore, the micro-applicator should optionally additionally be available in a kinkable and/or bendable embodiment. Hollow spaces or cavities in dentistry are subdivided into cavity classifications, such as I pits and fissures on the chewing surface (posterior teeth occlusally), II proximal surface of molars and premolars (posterior teeth approximally and possibly occlusally), III proximal surfaces of incisors and cuspids, excluding the incisal edge, IV as III, but including the incisal edge, V tooth neck surface, and VI defects in milk teeth.

In this context, the outer and optionally the inner diameter or cross section of the micro-container, and in particular the cross section of the inner shape of the application head, and the cross section or the diameter of the opening are dimensioned such that a liquid, in particular a liquid of a composition comprising organic solvents, and optionally at least one monomer and optionally water, automatically flows out of the internal space and thus out of the applicator when the applicator is held approximately vertically, and preferably wets the multitude of filaments, which in particular form a paintbrush. The volume of the internal space preferably contains maximally 1 ml, in particular from 0.02 to 0.9 ml, preferably from 0.1 ml to 0.9 ml.

The outer diameter (a) or the longest side of the cross section (a) of the application head amounts from 1.0 mm to 5.0 mm, in particular 1.0 mm to 4.5 mm, in particular from 1.0 mm to 4.0 mm, preferably from 2.0 mm to 4.0 mm, in particular each above-mentioned independently with +/−0.1 mm, particularly preferably about 3.5 mm +/−0.5 mm.

The length (b) of the outer side wall of the application head preferably amounts from 1 mm to 75 mm, in particular from 2 mm to 7.5 mm, preferably from 3 mm to 3.5 mm. The length (c) of the outer side wall of the micro-container preferably amounts from 2 mm to 75 mm, preferably 5 to 40 mm, in particular if the micro-container also assumes the function of a handhold, alternatively 5 mm to 6 mm may also be preferred. If the micro-container also serves as a handhold, it may be solid without internal space on the distal side.

The respective wall thickness of the side wall of the application head and/or of the micro container preferably amounts, each independently, from 0.1 mm to 1 mm, in particular from 0.2 to 0.6 mm, particularly preferably from 0.3 to 0.5 mm, preferably about 0.4 mm +/−0.05 mm.

The internal space of the micro container contains a volume of 0.04 ml to less than or equal to 1 ml, preferably with a volume of less than or equal to 0.75 ml, less than or equal to 0.5 ml, and is preferably intended to receive a liquid containing at least one organic, in particular volatile solvent, preferably a liquid dental composition.

According to a preferred alternative of the invention, the micro-container, in particular the tubular micro-container, is inserted, in particular slid in and/or turned in, inside the hollow application head, in particular the hollow tubular application head, in fit, in particular in transition fit or press fit, by distance (I₀), in particular by 6 mm, preferably from 2 mm to 5 mm. Furthermore, the tubular micro-container is insertable, in particular slidable in or turnable in, inside the hollow application head, in particular the tubular application head, in fit, in particular in transition fit or press fit, by distance (I₁) additionally to distance lo, in particular by at least 2 mm, preferably about at least 2 mm to 5 mm. The micro-container may be turned in according to an embodiment. The micro-container may preferably be made of an elastic or brittle material. According to a further alternative, a tubular micro-container may be turnable in, in particular screwable in, by a turning movement, in particular an axial rotational movement, preferably a screwing movement, inside the hollow application head in transition fit or press fit by distance (I₁) additionally to distance I₀.

The internal space of the sleeve-like micro-container is closed by a film or fragile layer at its proximal front face. The film or the fragile layer is pierced by the at least one punching spike so that the liquid may escape from the internal space of the micro-container. The fragile layer may also be destroyable by the action of an external force on the application head.

According to another particularly preferred embodiment, the ratio of distance I₁, by which the micro-container is slidable in (distance I₁) or turnable in inside the hollow application head in transition fit or press fit additionally to distance I₀, to the length c of the outer side wall of the micro-container is 1 to 2 to 1 to 75, preferably 1 to 5 to 1 to 25.

According to a preferred embodiment, the length (b) of the outer side wall of the application head has a ratio of 1 to 1.2 to 1 to 25, in particular of 1 to 1.3 to 1 to 6, preferably of 1 to 1.5 to 1 to 3, to the length (c) of the outer side wall of the micro-container (b to c). In addition, the averaged length (e) of the respective punching spike, optionally with base, preferably has a ratio of 1 to 2 to 1 to 20, preferably of 1 to 2.5 to 1 to 10, to the length (c) of the outer side wall of the micro-container as ratio of b to c. The punching spike according to the invention is explicitly not formed as being a displacement piston, but serves solely to destroy the film or the fragile layer.

The punching spike may have different geometries; thus the punching spike may be a single pin with a tip and may be arranged next to the opening in the front face. Alternatively, the punching spike with tip may have a proximal base, by which the punching spike is fixed in the inner shape of the sleeve-like application head in the proximal region of the application head by means of a fit, in particular with transition or press fit, of the outer shape of the base. Preferably, the punching spike has a conduit, being continuous from distal to proximal, with cross section (d₄), in particular the cross section amounts from 0.2 mm² to 4 mm², preferably from 0.4 mm² to 1 mm². Particularly preferably, the punching spike with base also has a respective continuous conduit. Alternatively, the respective punching spike may have at least one conduit, preferably two to 20 conduits, being continuous from distal to proximal, with total cross section across all channels (d₄), in particular this cross section amounts from 0.2 mm² to 4 mm², preferably from 0.4 mm² to 1 mm². According to a further alternative, the base may have two to 20 punching spikes each having a channel. In these to alternative, the base also has a corresponding number of two to 20 accordingly continuous conduits in the alternatives a) base with punching spike and two to 20 conduits in the punching spike or b) base with two to 20 punching spikes each having a conduit.

According to another alternative embodiment, the punching spike has a distal tip. In an alternative, the tip may be formed by a curved plane, preferably a concave or planar plane, in which the punching spike, in particular the punching spike with or without conduit, is formed at the distal end. Preferably, the spike has a cutting edge at the tip. The same applies to the respective punching spike if there are several punching spikes.

Furthermore, the micro-container may be bent, U-shaped and/or formed as handhold at its distal side. Preferably, the handhold has circumferential groove-like recesses or knobs. According to a further preferred embodiment, the distal side of the tubular container may be welded and/or pressed.

The micro-applicator has a multitude of filaments at the front face in the proximal region of the application head, in particular for the formation of a paintbrush. The preferred minimum length (f) of the filaments amounts from 2 mm to 8 mm, in particular from 3 mm to 7 mm, preferably from 4 mm to 6 mm. Preferably, the diameter of the filaments, each independently, amounts from in the distal region 0.2 mm to 0.4 mm, in particular 0.3 mm +/−0.25 mm, and optionally in the proximal region at the tip 0.05 mm to 0.3 mm, in particular 0.2 mm with +/−0.1 mm. In this context, the filaments are preferably designed in such a way that the multitude of the filaments for the formation of a paintbrush exert a capillary action on the liquid or paste so that a liquid being stored in the micro-container is transferred in the application region after destroying the film of the fragile layer when the applicator, in particular the application head, is approximally vertically oriented, in particular in an angle of 70 to 110°, preferably 80 to 200° with respect to the horizontal, to make the liquid applicable with the paintbrush.

In a particularly preferred alternative, the inner shape of the hollow sleeve-like application head is formed in fit to the outer shape of the micro-container, in particular the tubular micro-container accordingly in such a way that the micro-container is slidable in into the application head until the inner proximal front face, in particular until the front face of the side walls, or the film or fragile layer of the micro-container, in particular of the tubular micro-container, abuts in the inside against the base of the punching spike or against the proximal inside wall of the application head, wherein a) the angle alpha (α) between the central axis in the distal plane at the distal region of the hollow sleeve-like application head and the longitudinal central axis in the proximal region amounts to alpha less than or equal to 180°, in particular the angle alpha amounts from 175° to 110°, in particular from 170° to 130°, or b) the micro-container, in particular the tubular micro-container, has a predetermined kink point, in particular the container or the side wall of the micro-container at this point is thinner compared with the remaining container or the remaining side wall, or the distal region of the micro-container, in particular of the tubular micro-container, which protrudes distally into the application region after sliding in the micro-container, is compressible or compressible and bendable. Preferably, the distally protruding region of the micro container is compressible and/or bendable after destroying the film or the fragile layer. The proximal inside wall, in particular wall, of the application head preferably has the opening at the front face of the proximal region. The opening in the front face of the proximal region may preferably have the same cross section as the at least one conduit in the punching spike and optionally in the base.

A subject matter of the invention is further an application, in which i) the film or the destroyable layer is selected from one of the following materials, comprising metals, such as aluminum, alloys, organic polymers, such as PET (polyethylene terephthalate), PE (polyethylene), PP (polypropylene), COC (cyclic olefin copolymer), COP (cyclic olefin polymer), acrylonitrile butadiene rubber, ethylene vinyl alcohol polymer (EVOH, EVAL), halogenated polymers, chloroprene rubber, perfluorinated rubber, HDPE, LDPE, porous expanded polypropylene (PEPP) and/or expanded polypropylene (EPP), PA (polyamide), glass, hybrid material or a composite material of the afore-mentioned materials, in particular the film or the layer is a welded or laminated aluminum film, COC, COP, PP, EVOH or PET film, and/or

ii) the application head is selected from one of the following materials, comprising thermoplastic elastomers, such as copolyamide, polyester elastomers, PP/EPDM (EPDM: ethylene propylene diene rubber), styrene block copolymers, such as SBS, SEBS, SEPS, SEEPS and MPs, urethane-based thermoplastic elastomers, polyethylene, polypropylene, silicone, polyamide (PA) or rubber, in particular the application head and the filaments are made of the same material, preferably application head and filaments consist of the same material and are formed as an integral component, and/or

iii) the material of which the micro-container is selected, comprises polyethylene, polypropylene, ethyl vinyl alcohol polymers, cyclic olefin polymers (COP) and/or cyclic olefin copolymers (COC), particularly preferably copolymers of ethane with cyclic olefins, such as norbornene, norbornene derivative, tetracyclododecene, or polymers of cyclooctene, halogenated polymers, HDPE, LDPE, porous expanded polypropylene (PEPP) and/or expanded polypropylene (EPP) and/or metallic films or a composite mateiral of the afore-mentioned materials, and/or

iv) the material of the punching spike and optionally of the base, each independently, is selected from one of the following materials, comprising metal, alloy, glass, ceramic, hybride material or plastic having an elastic modulus of greater than or equal to 1500 MPa according to ISO 527 or greater than or equal to 1300 MPa according to ISO 178.

The application head and/or the micro-container may preferably be made of an inert polymer, in particular a thermoplastic polymer, comprising cyclic olefin polymers (COP), PE, PP, HDPE, LDPE, porous expanded polypropylene (PEPP) and/or expanded polypropylene (EPP). According to the invention, the application head and/or the micro-container made of thermoplastic polymer may additionally have a barrier made of EVOH. The application head and/or the micro-container and/or the filaments may preferably made of polyamide or also of an iner polymer, in particular a thermoplastic polymer, comprising cyclic olefin polymers (COP), PE, PP, HDPE, LDPE, porous expanded polypropylene (PEPP) and/or expanded polypropylene (EPP).

In the present case, rubber is understood to mean natural or synthetic rubber, such as, amongs others, styrene butadiene rubber, butadiene rubber (BR), acrylonitrile butadiene rubber (NBR), butyl rubber (IIR) ethylene propylene diene rubber (EPDM), chloroprene rubber (CR) and polyisoprene rubber (IR).

In an alternative, it may be conceivable for at least one brush, sponge, flocking, ridge, ball, paintbrush, spatula, mixing pad and/or porous means to be arranged in the application region additionally to the filaments.

The material of which the container and optionally the film or the destroyable layer consists of may have in an alternative, preferably for COP and/or COC a flexural modulus of 1800 to 2200 MPa (ISO178), in particular about 2200 MPa +/−100 MPa, and/or a flexural strength of 80 MPa to 100 MPa, preferably of 94 MPa +/−5 MPa (ISO 178), and a tensile modulus of 2200 MPa to 2600 MPa, in particular of 2400+/−100 MPa (ISO527), and/or a tensile strength of 40 MPa to 70 MPa, in particular of 60 MPa +/−5 MPa (ISO527), and/or a tensile elongation of 10 MPa to 30 MPa, in particular of 20 MPa (ISO527).

Furthermore, a subject matter of the invention is a kit comprising a micro-applicator, wherein a liquid is contained in the micro-container, in particular the tubular micro-container, in particular a liquid containing at least one organic solvent, preferably a liquid dental composition comprising at least one volatile organic solvent and optionally at least one monomer as well as optionally water, and/or optionally an organic acid.

Likewise a subject matter of the invention is a kit comprising an outer packaging as well as a multitide of micro-applicators. Preferably, the liquid dental composition is an adhesive, a light-curing adhesive, an etching agent, a dental adhesive agent.

Another subject matter of the invention is an applicator, in which the internal space is filled with a dental liquid, in particular with a dental adhesive, preferably with a self-etching dental adhesive, such as iBOND® Self Etch. Another subject matter of the invention is the use of an applicator according to the invention for application of liquid dental compositions, such as in particular iBOND® Self Etch on teeth. Another subject matter of the invention is a kit containing a self-etching dental adhesive for use in the treatment of a demineralization of a tooth or teeth and/or in the treatment of caries, preferably the initial caries and/or Caries profunda.

According to an embodiment variant, a subject matter of the invention is a kit comprising at least one applicator, wherein each applicator is individually stored in a lightproof package.

Adhesives according to the invention being stored in the internal space of the applicator are preferably self-etching dental adhesives.

The dental composition, in particular the dental adhesive, preferably comprises an A) radically polymerisable monomer having an acid group in the molecule, B1) optionally a photosensitizer, and/or optionally B2) a peroxide, C) a water-miscible solvent, such as alcohol, ketone, ester, ketal, isopropylidene glycerin, ethanol, preferably acetone, and D) water.

The kit may further comprise E) at least one radically polymerisable monomer without an acid group, preferably not or hardly being soluble in water (less than 2 g/100 ml H₂O).

The radically polymerisable monomers having an acid group in the molecule according to A), also referred to as acidic component, comprise polymerisable monomers having at least one, preferably several ethylene groups as well as at least one carboxylic acid group, carboxylic anhydride group, phosphoric acid group and/or sulfonic acid group.

Monofunctional polymerisable monomers having a carboxylic acid or carboxylic acid anhydride group in the molecular may be selected from monocarboxylic acids, dicarboxylic acids, tricarboxylic acids, tetracarboxylic acids, polycarboxylic acids and anhydrides thereof. Preferred compounds may include carboxylic acids and/or anhydrides, such as maleic acid, p-vinyl benzoic acid, 11-(meth)acryloyloxy 1,1-undecane dicarboxylic acid (MAC-10), 1,4-di(meth)acryloyloxy ethyl pyromellitic acid, 6-(meth)acryloyloxy ethyl naphthalene 1,2,6-tricarboxylic acid, 4-(meth)acryloyloxy methyl trimellitic acid and anhydrides thereof, 4-(meth)acryloyloxy ethyl trimellitic acid and the its anhydride, 4-(meth)acryloyloxy butyl trimellitic acid and its anhydride, 4-[2-hydroxy-3-(meth)acryloyloxy] butyl trimellitic acid and its anhydride, 2,3-bis(3,4-dicarboxybenzoyloxy) propyl (meth)acrylate, 2-, 3-, or 4-(meth)acryloyloxy benzoic acid, N-O-di(meth)acryloyloxy tyrosine, O-(meth)acryloyloxy tyrosine, N-(meth)acryloyloxy tyrosine, N-(meth)acryloyloxy phenylalanine, N-(meth)acryloyl-p-amino benzoic acid, N-(meth)acryloyl-O-amino benzoic acid, Adduct made of glycidyl (meth)acrylate with N-phenyl glycine or N-tolyl glycine, 4-[(2-hydroxy-3-(meth)acryloyloxy propyl) amino] phthalic acid, 3- or 4-[N-methyl-N-(2-hydroxy-3-(meth)acryloyloxy propyl) amino] phthalic acid, (meth)acryloyl amino salicylic acid and (meth)acryloyloxy salicylic acid. 11-(meth)acryloyloxy-1,1-undecane dicarboxylic acid (MAC-10) and 4-methacryloyloxy ethyl trimellitic acid (4-MET) or their anhydrides (4-META) are preferred. Polyfunctional polymerisable monomers having at least two carbonyl groups in the molecule, which are usable as component A) may comprise dicarboxylic acids, tricarboxylic acids, tetracarboxylic acids and derivatives thereof, such as, for example, an addition product made of 2-hydroxyethyl (meth)acrylate and pyromellitic dianhydride (PMDM), an addition reaction product made of 2 hydroxyethyl (meth)acrylate and 1 mole maleic acid anhydride or 3,3′,4,4′-benzophenone tetracarboxylic acid anhydride (BTDA) or 3,3′,4,4′-biphenyl tetracarboxylic acid anhydride, and 2-(3,4-dicarboxybenzoyloxy) 1,3-di(meth)acryloyloxy propane.

Polymerisable monomers having at least one phosphoric acid group in the molecule comprise, for example, 2-(meth)acryloyloxy ethyl acid phosphate, 2- and 3-(meth)acryloyloxy propyl acid phosphate, 4-(meth)acryloyloxy butyl acid phosphate, 6-(meth)acryloyloxy hexyl acid phosphate, 8-(meth)acryloyloxy octyl acid phosphate, 10-(meth)acryloyloxy decyl acid phosphate, 12-(meth)acryloyloxy dodecyl acid phosphate, bis(2-(meth)acryloyloxy ethyl) acid phosphate, bis({2 or 3}-(meth)acryloyloxy propyl) acid phosphate, 2-(meth)acryloyloxy ethyl phenyl acid phosphate, 2-(meth)acryloyloxy ethyl p-methoxy phenyl acid phosphate and the like. The phosphoric acid in these compounds may be substitued by a thiophosphoric acid group.

The following are preferred of the above-mentioned monomers 2-(meth)acryloyloxy ethyl phenyl acid phosphate and 10-(meth)acryloyloxy decyl acid phosphate. The monomers having a phosphoric acid group may be used alone or in conjunction.

Polymerisable monomers having a sulfonic acid group in the molecule may comprise 2-sulfoethyl (meth)acrylate, 2- or 1-sulfo-1- or -2-propyl (meth)acrylate, 1- or 3-sulfo-2-butyl (meth)acrylate, 3-bromine-2-sulfo-2-propyl (meth)acrylate, 3-methoxy-1-sulfo-2-propyl (meth)acrylate, 1,1-dimethyl 2-sulfoethyl (meth)acrylamide and 2-methyl 2-(meth)acrylamide propane sulfonic acid, 2-methyl 2-(meth)acrylamide propane sulfonic acid is preferred.

The polymerisable monomer A) may comprise an acid group which is present as salt, such as, for example as monovalent or polyvalent metal salt or ammonium salt. In this context, it is however preferred for the monomer A) to act as an acid when used in conjunction with another acidic compound and contacted with the other acidic component. The above components A) may be used alone or in conjunction with each other.

Preferred components B1) and/or B2) comprise as B1) optionally a photosensitizer, and/or optionally B2 a peroxide.

Preferred components B1) comprise an alpha-ketocarbonyl compound or acyl phosphine oxide compound. Alpha-diketone, alpha-ketoaldehyde, alpha-ketocarboxylic acid, alpha-ketocarboxylate are concretely preferred. Alpha-diketones, such as for example, diacetyl, 2,3-pentanedione, 2,3-hexanedion, benzyl, 4,4′-dimethoxy benzyl, 4,4′-diethoxy benzyl, 4,4′-oxybenzyl, 4,4′-dichlorbenzyl, 4-dinitrobenzyl, alpha-naphthyl, camphor quinone, camphor quinone sulfonic acid, camphor quinone carboxylic acid and 1,2-cyclohexanedion, alpha-ketoaldehydes, such as, for example methylglyoxal, and phenylglyoxal, and others, such as, for example pyruvic acid, benzoyl formic acid, phenyl pyruvic acid, methyl pyruvate, ethyl benzoyl formate, methyl phenyl pyruvate and butyl phenyl pyruvate, are concretely preferred. Particularly preferred are alpha-diketones due to their stability, as well as diacetyl, benzyl and camphor quinone.

Furthermore, component B1) comprises benzoyl dimethoxy phosphine oxide, benzoyl ethoxy phenyl phosphine oxide, 2-methyl benzoyl diphenyl phosphine oxide, 2,4,6-trimethyl benzoyl diphenyl phosphine oxide as well as the derivatives thereof. All photoactive compouns may be used alone or in mixture.

Compounds, such as diacetyl diperoxide, dipropyl peroxide, dibutyl peroxide, dicapryl peroxide, dilauryl peroxide, benzoyl peroxide (BPO), p,p′-dichlorbenzoyl peroxidw, p,p′-dimethoxy benzoyl peroxide, p,p′-dimethyl benzoyl peroxide and p,p′-dinitrodibenzoyl peroxide, and inorganic peroxides, such as, for example ammonium persulfate, potassium persulate, potassium chlorate, potassium bromate and potassium perphosphate may be used as peroxide B2). BPO is preferred.

Alcohol, ketones, esters, ketals, isopropylidene glycerin, such as ethanol, but preferably acetone, may preferably be used as component C) a water-miscible solvent.

Component C) according to the invention is a water-soluble organic solvent. This is used to uniformly dissolve or disperse each of the above components; the solvent should be inert to the components and advantageously volatile. Higher alcohols, such as, for example, ethylene glycol, propylene glycol and glycerin may also be used.

Component E) preferably comprises at least one radically polymerisable monomer without acid group, in particular not or hardly being soluble in water. Preferred radically polymerisable monomers without acid group, being different from component (A), comprise aromatic vinyl compounds, such as, for example styrene and divinyl benzene, vinyl ester, such as, for example, vinyl acetate, aliphatic esters of (meth)acrylic acid, such as, for example, methyl (methacrylate, ethyl (meth)acrylate, propyl (meth)acrylate, butyl (meth)acrylate, pentyl (meth)acrylate, hexyl (meth)acrylate, neopentyl glycol di(meth)acrylate and trimethylolpropane tri(meth)acrylate, aromatic esters, such as, for example, phenyl (meth)acrylate, aromatic (meth)acrylates, such as, for example 2-hydroxy-3-phenoxy propyl (meth)acrylate, an adduct made of 1 mole bisphenol-A with 2 mole glycidyl (meth)acrylate (Bis-GMA), a condensate made of 1 mole of an addition product of bisphenol-A with glycidyl ether and of an addition polymer made of bisphenol-A with glycidyl ether and 2 mole (meth)acrylic acid and a condensate made of 1 mole of an adduct made of bisphenol A with ethylene oxide and 2 mole (meth)acrylic acid (number of the addition chain of ethylene oxide m+n=2.6), urethane bond-containing methacrylates, such as, for example 2-(meth)acryloyloxy ethyl isocyanate and an adduct (UDMA) made of 2 mole hydroxyl ethyl (meth)acrylate with 1 mole 2,2,4- (or 2,4,4-) trmethyl-1,6-hexamethylene diisocyanate, aliphatic (meth)acrylic acid esters, such as, for example, 1,6-hexyamethylene dimethacrylate (1,6-HX), neopentyl glycol di(meth)acrylate and trimethylolpropane tri(meth)acrylate, polyethylene glycol di(meth)acrylates (chain length n=less than 6), such as, for example, ethylene glycol di(meth)acrylate, diethylene glycol di(meth)acrylate and triethylene glycol di(meth)acrylate, and polypropylene glycol di(meth)acrylates (chain length n=12 or less), such as, for example propylene glycol di(meth)acrylate, dipropylene glycol di(meth)acrylate, tripropylene glycol di(meth)acrylate and nanopropylene glycol di(meth)acrylate. Components E) according to the invention comprise monomers comprising at least one (meth)acrylic group selected from methyl methacrylate, ethyl methacrylate, propyl methacrylate, butyl methacrylate, n-hexyl methacrylate, 2-phenoxy ethyl methacrylat, isobornyl methacrylate, isodecyl methacrylate, polypropylene glycol monomethacrylate, tetrahydrofuryl methacrylate, methyl acrylate, ethyl acrylate, propyl acrylate, butyl acrylate, n-hexyl acrylate, 2-phenoxy ethyl acrylate, isobornyl acrylate, isodecyl acrylate, polypropylene glycol monoacrylate, tetrahydrofuryl acrylate, hydroxyethyl acrylate, hydroxypropyl acrylate, hydroxyethyl methacrylate, hydroxypropyl methacrylate, or mixtures thereof, or at least one crosslinker. Typical crosslinker include BDMA, 1,4-butanediol dimethacrylate (1,4-BDMA) or pentaerythritol tetraacrylate, urethane dimethacrylate (UDMA), bis-GMA monomer (Bisphenyl-A glycidyl methacrylate). The use of diluents (highly fluid acrylates, such as triethylene glycol dimethacrylate (TEGDMA) and diethylene glycol dimethacrylate (DEGMA)), etc.

Preferred (meth)acrylates having at least two (meth)acrylic groups are selected from ethanediol dimethacrylate, tetraethylene glycol dimethacrylate, diethylene glycol dimethacrylate, ethylene glycol dimethacrylate, polyethylene glycol dimethacrylate (400) or (600), butanediol dimethacrylate, hexanediol dimethacrylate, decanediol dimethacrylate, dodecanediol dimethacrylate, 1,3-butylene glycol dimethacrylate, dipropylene glycol methacrylate, bisphenol-A dimethacrylate, bisphenol-A dimethacrylate derivatives, such as ethoxylated 2-bisphenol-A dimethacrylate, trimethylolpropane trimethacrylate, triethylene glycol dimethacrylate, 2,2-bis-(4-(3-methacryloxy-2-hydroxypropoxy) phenyl) propane (bis-GMA), tricyclodecane dimethanol dimethacrylate, an urethane methacrylate having at least two methacrylic groups or a mixture containing at least one of the (meth)acrylates.

Preferred (meth)acrylates having three to six methacrylic groups are selected (i) with three (meth)acrylic groups, from ethoxylated 15-trimethylolpropane triacrylate, ethoxylated 5-pentaerythritol triacrylate, propoxylated 5.5-glycerol triacrylate, trimethylolpropane trimethacrylate, tris(2-hydroxyethyl) isocyanurate triacrylate, and/or (ii) with four (meth)acrylic groups, from ditrimethylolpropane tetraacrylate, ethoxylated 4-pentaerythritol tetraacrylate, pentaerythritol tetraacrylate, ditrimethylolpropane tetramethacrylate, ethoxylated 4-pentaerythritol tetramethacrylate, pentaerythritol tetramethacrylate, and/or (iii) with five (meth)acrylic groups, from dipentaerythritol pentaacrylate, i-pentaerythritol pentamethacrylate, dipentaerythritol pentamethacrylate, di(tetramethylolmethane) pentamethacrylate, and/or (iv) with six (meth)acrylate groups, from a dipentaerythritol hexa(meth)acrylate. Oligomers of (meth)acrylate, in particular urethane diacrylate oligomer, are suitable as well.

In the following, the invention is clarified by means of examples without being limited to these examples:

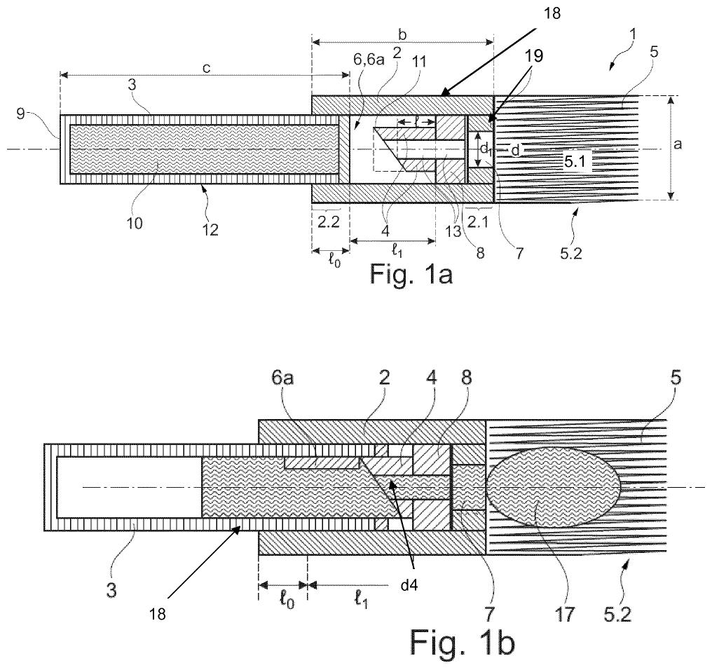

FIGS. 1a and 1b show a dental micro-applicator 1 having a hollow sleeve like application head 2 which has a distal 2.2 and a proximal region 2.1 as well as an application region 5.1 at the front face at the proximal region of the application head 2. A punching spike 4, the tip 11 of which is oriented toward distal, is arranged in the proximal region 2.1 in the application head 2. The punching spike may be fixed in place by a fit or may be formed integrally with the application head. An opening 7 having a diameter d1 is formed in the front face for passage of a dental, liquid composition. The punching spike 4 has a conduit having a diameter d4. The micro-container 3 is slid in from distal into the application head 2 and seals the two components against each other. The dental composition is tightly sealed in the internal space 10 against the atmosphere by a film 6a at the proximal front face 6. The micro-container 3 is slid in inside the hollow tubular application head 2 in transition fit or press fit by distance I₀ and may additionally be slid in inside the hollow tubular application head 2 in transition fit or press fit by distance I₁. The punching spike 4 with tip 11, and cutting edge preferably, has a proximal base 8. The punching spike 4 shown has a conduit 13 with cross section d₄, being continuous from distal to proximal. In FIG. 1b, the film 6a is opened and the liquid can escape as drops 17 through the opening 7 into the filaments. The micro-applicator may alternatively have a punching spike with several conduits or several punching spikes each having at least one conduit.

The micro applicator is formed as handhold at its distal side 9. Furthermore, FIG. 1a shows the length a of the cross section of the application head, the length b of the outer side wall 18 of the application head 2 as well as the length c of the outer side wall 12 of the micro-container 3 and also the averaged length e of the punching spike.

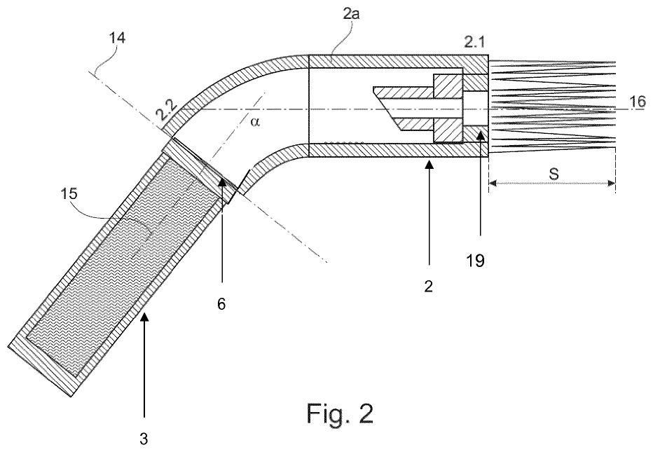

FIG. 2 shows a micro-applicator, the inner shape of the hollow sleeve-like application head 2 is formed in fit to the outer shape of the tubular micro-container 3 so that the micro-container 3 can be slid in into the application head 2 until the proximal front face 6 of the tubular container 3 abuts in the inside against the base 8 of the punching spike or against the proximal inside wall 19 of the application head 2, wherein a) the angle alpha (α) between the central axis 15 in the distal plane 14 at the distal region 2.2 of the hollow sleeve-like application head 2 and the longitudinal central axis 16 in the proximal region 2.1 amounts to alpha less than or equal to 180°, particularly less than than 180°, preferably the angle alpha mounts from 175° to 110°.

### LIST OF REFERENCE NUMERALS

1 micro-applicator
2 application head, in particular hollow sleeve-like application head
2.1 proximal region
2.2. distal region
3 micro-container, in particular tubular micro-container
4 punching spike
5 filament
5.1 application region
5.2. paintbrush
6 proximal front face
6a film
6b layer, in particular fragile layer
7 opening
8 base
9 distal side
10 internal space
11 tip
12 side wall of the micro-container
13 conduit
14 distal plane
15 central axis
16 longitudinal central axis
17 drop of the liquid
18 side wall of the application head
19 proximal inside wall, in particular wall, of the application head
a length of the cross section of the application head
b length of the outer side wall 17 of the application head 2
c length of the outer side wall 12 of the micro-container 3
e averaged length of the punching spike
d1 cross section of the opening 7
d4 cross section d₄ of the conduit in the punching spike
[symbol] or I₀ distance in the inside by which the micro-container is inserted, in particular slid in or turned in, into the hollow sleeve-like application head 2 in transition fit or press fit in storage position
[symbol] or I₁ distance by which the micro-container is inserted, in particular slidable in or turned in, inside the hollow sleeve-like application head 2 in transition fit or press fit additionally to distance I₀

1. A dental micro-applicator (1) having a hollow sleeve-like application head (2) with a distal (2.2) and a proximal (2.1) region and with an application region (5.1) at the front face of the proximal region (2.1) of the application head (2),
wherein
at least one punching spike (4), the tip (11) of which is oriented toward distal, is arranged in the hollow sleeve-like application head (2) in the proximal region (2.1), and an opening (7) is formed at the front face of the proximal region (2.1) for passage of a liquid or paste to be applied, a micro-container (3), the outer shape of which corresponds to the positive of the inner shape of the application head (2) being formed as the negative, being inserted, in particular slid in or turned in, from distal into the hollow sleeve-like application head (2), and the micro-container (3) having an internal space (10) to receive the liquid or paste to be applied, and the micro container (3) being closed at its distal side (9) and at its proximal front face (6), wherein
the punching spike (4) with tip (11) has a proximal base (8), by which the punching spike (4) is fixed in the inner shape of the sleeve-like application head (2) in the proximal region of the application head (2) by means of a fit with transition fit or press fit of the outer shape of the base (8), wherein the punching spike (4) has a conduit (13), being continuous from distal to proximal, with cross section (d₄), wherein the cross section amounts from 0.2 mm² to 4 mm², wherein the punching spike (4) with base (8) also has a respective continuous conduit.

2. The applicator according to claim 1, wherein the micro-container (3) is inserted, in particular slid in or turned in, inside the hollow application head (2) in transition fit or press fit by distance (I₀).

3. The applicator according to claim 2, wherein a) the micro-container (3) is slidable in inside the hollow application head (2) in transition fit or press fit by distance (I₁) additionally to distance (I₀), or b) a tubular micro-container is turnable in by a turning movement inside the hollow application head (2) in transition fit or press fit by distance (I₁) additionally to distance (I₀).

4. The applicator according to claim 1, wherein the micro-container (3) is closed by a film (6a) or a fragile layer (6b) at its proximal front face (6).

5. The applicator according to claim 1, wherein the length (b) of the outer side wall (17) of the application head (2) to the length (c) of the outer side wall (12) of the micro-container (3) amount to 1 to 1.2 to 1 to 25.

6. The applicator according to claim 1, wherein the inner shape of the hollow sleeve-like application head (2) is formed in fit to the outer shape of the micro-container (3) so that the micro-container (3) is slidable in or turnable in into the application head (2) until the proximal front face (6) of the micro container (3) abuts in the inside against the base (8) of the at least one punching spike (4) or against the proximal inside wall (19) of the application head (2).

7. The applicator according to claim 4, wherein the film (6a), or fragile layer (6b) at the proximal side of the micro-container (3), in particular of a tubular micro-container, is destroyed or cut off by at least one punching spike (4) when sliding in or turning in the micro-container into the application head (2).

8. The applicator according to claim 1, wherein a multitude of filaments (5), in particular having a minimal length (f) of 2 mm to 8 mm, in particular 3 mm to 7 mm, preferably 4 mm to 6 mm is formed in the application region (5.1) at the front face in the proximal region of the application head (2) to form a paintbrush (5.2).

9. The applicator according to claim 8, wherein the micro-container is a hollow section closed at the proximal and distal front face.

10. The applicator according to claim 1, wherein
a) the micro-container is inserted from distal into the hollow sleeve-like application head (2), the outer shape of the micro-container (3) being formed in fit to the outer shape of the application head (2), or
b) the micro-container is inserted from distal into the hollow sleeve-like application head (2), the outer shape of the micro-container (3) being spaced by a seal from the inner shape of the application head (2).

11. The applicator according to claim 1, wherein the inner shape of the hollow sleeve-like application head (2) is formed in fit to the outer shape of the micro-container (2) so that the micro-container (3) is slidable in or turnable in into the application head (2) until the proximal front face (6) of the micro-container (3) abuts in the inside against the base (8) of the at least one punching spike or against the proximal inside wall of the application head (2), wherein a) the angle alpha (α) between the central axis (15) in the distal plane (14) at the distal region (2.2) of the hollow sleeve-like application head (2) and the longitudinal central axis (16) in the proximal region (2.1) of the application head amounts to alpha less than or equal to 180°, in particular the angle alpha amounts from 175° to 110°, or b) the micro-container (3) has a predetermined kink point.

12. The applicator according to claim 1, wherein
i) the film or the fragile layer is selected from one of the following materials, comprising metals, such as aluminum, alloys, PET (polyethylene terephthalate), PE (polyethylene), PP (polypropylene), COC (cyclic olefin copolymer), COP (cyclic olefin polymer), acrylonitrile butadiene rubber, ethylene vinyl alcohol polymer (EVOH, EVAL), halogenated polymers, chloroprene rubber, perfluorinated rubber, PA (polyamide), HDPE, LDPE, porous expanded polypropylene (PEPP) and/or expanded polypropylene (EPP), glass, hybrid material or a composite material of the afore-mentioned materials, in particular the film or the layer is a welded or laminated aluminum film, COC, COP or PET film, and/or
ii) the application head is selected from one of the following materials, comprising thermoplastic elastomers (TPE), silicone, polyamide, polyethylene, polypropylene, rubber, or a composite material of the afore-mentioned materials, in particular the application head and the filaments are made of the same material, and/or
iii) the material of the micro-container (3) is selected from polyethylene, polypropylene, ethyl vinyl alcohol polymers, cyclic olefin polymers (COP) and/or cyclic olefin copolymers (COC), particularly preferably copolymers of ethane with cyclic olefins, such as norbornene, norbornene derivative, tetracyclododecene, or polymers of cyclooctene, halogenated polymers, HDPE, LDPE, porous expanded polypropylene (PEPP) and/or expanded polypropylene (EPP) and/or metallic films, and/or
iv) the material of the at least one punching spike (4) and optionally of the base (8), each independently, is selected from one of the following materials, from plastic having an elastic modulus of greater than or equal to 1700 MPa according to ISO 527 or greater than or equal to 1300 MPa according to ISO 178, metal, alloy, glass, ceramic, hybrid material.

13. A kit comprising a micro-applicator according to claim 1, a liquid being contained in the micro-applicator (3), in particular a liquid containing at least one organic solvent, preferably a liquid dental composition comprising at least one volatile organic solvent and optionally at least one monomer, and optionally water, and/or optionally an organic acid.

14. The kit according to claim 13, comprising an outer packaging as well as a multitude of micro-applicators.

15. Use of a micro-applicator according to claim 1, or of a kit according to claims 13 for application of liquid dental compositions.
